(19) United States
(12) Patent Application Publication
(10) Pub. No.: US 2021/0348227 A1
(43) Pub. Date: Nov. 11, 2021

(54) SUBSTRATE FOR NUCLEIC ACID ANALYSIS, FLOW CELL FOR NUCLEIC ACID ANALYSIS, AND IMAGE ANALYSIS METHOD

(71) Applicant: HITACHI HIGH-TECH CORPORATION, Tokyo (JP)

(72) Inventors: Noriko Baba, Tokyo (JP); Masatoshi Narahara, Tokyo (JP); Naoshi Itabashi, Tokyo (JP); Toru Yokoyama, Tokyo (JP)

(73) Assignee: HITACHI HIGH-TECH CORPORATION, Tokyo (JP)

(21) Appl. No.: 17/276,898

(22) PCT Filed: Dec. 24, 2019
(86) PCT No.: PCT/JP2019/050512

§ 371 Date: Mar. 17, 2021

(30) Foreign Application Priority Data
Jan. 9, 2019 (JP) 2019-001562

(51) Int. Cl. C12Q 1/6874; G06T 7/33; G06T 7/32; G06T 7/00

(52) U.S. Cl. CPC C12Q 1/6874; G06T 7/337; G06T 2207/30072; G06T 7/0012; G06T 7/32

(57) ABSTRACT

At the positions of spots which are arranged on a substrate, image aligning is made difficult by the occurrence of a recognition error of the positions of spots, said spots being adjacent to each other in a patterned form, or a displacement caused by the expansion or deformation of the substrate due to device operation, temperature control, etc. The present invention provides: a substrate for nucleic acid analysis, on the surface of which a patterned spot area provided with spots to which a biopolymer is adhered and a randomly distributed spot area are formed; and an analysis method.

### TECHNICAL FIELD

The present invention is related to a substrate for nucleic acid analysis, a flow cell for nucleic acid analysis, and an image aligning method and related to the arrangement of a patterned spot area and a randomly distributed spot area for analysis to measure biological substances.

### BACKGROUND ART

Recently, in a nucleic acid analyzer, a large amount of base sequence information can be sequenced simultaneously in parallel. Nucleic acids as an analysis target are immobilized on a substrate, and a sequence reaction is repeated. A technique of incorporating fluorescent nucleotide for identifying a base into a base sequence of a nucleic acid to specify the base based on fluorescent bright points emitted from the fluorescent nucleotide is used. Images corresponding to a plurality of bases of nucleic acids are provided from the analyzer. In a sequence unit called one cycle, each of portions of the immobilized nucleic acids corresponding to one base is sequenced. By repeating this cycle, bases of each nucleic acid can be sequenced in order. In order to acquire a large amount of base sequence information, it is necessary to increase the density of nucleic acids immobilized on a substrate. Examples of the kind of the substrate on which nucleic acids are immobilized include a substrate including randomly distributed spots on which nucleic acids are randomly immobilized and a substrate including patterned spots on which nucleic acids are arrayed and immobilized in a patterned form. When immobilized nucleic acids are adjacent to each other, randomly distributed spots cannot be detected separately. When nucleic acids are arranged with high density, patterned spots are effective. For example, in a substrate for analysis disclosed in PTL 1, patterned spots where attachment spots to which nucleic acids are bound are arranged in a grid shape on a substrate are formed to implement high density.

In a method of analyzing nucleic acids on this substrate, it is necessary to accurately identify positions of individual spots in a fluorescent image as bright points. In general, even in fluorescent images obtained by imaging the same detection field of view, if there is a movement such as stage driving or the like for changing the field of view, the imaged position may be displaced to a different position due to the accuracy of driving control. Therefore, coordinate positions of one spot may be imaged as different coordinate positions in the individual images. In order to accurately identify positions of individual spots, it is necessary to accurately acquire coordinate positions of individual spots on a substrate.

Even in a case where the patterned spots are formed to implement high density as disclosed in PTL 1, when displacement caused by a recognition error occurs, it is difficult to identify positions of attachment spots of nucleic acids because the attachment spots are periodically arrayed. Therefore, PTL 2 discloses an analysis method including: deleting some attachment spots among the patterned attachment spots on the substrate; and detecting deletion portions to correct displacement.

### CITATION LIST

### Patent Literature

PTL 1: US2009/0270273A

PTL 2: US8774494B

### SUMMARY OF INVENTION

### Technical Problem

In order to acquire a large amount of base sequence information, when attachment spots of patterned samples are arranged on a substrate for increasing the density of the samples, the density of the samples increases. However, since the attachment spots are periodically arrayed, there is a problem in that it is difficult to distinguish between positions of attachment spots adjacent to each other. In addition, even when a sequence reaction is repeated on nucleic acids immobilized on a substrate, positions of the nucleic acids immobilized on the substrate do not change. However, an image at completely the same position may not be acquired per cycle due to the driving accuracy of a stage with the substrate placed thereon, the expansion or deformation of the substrate caused by a temperature control system, or the like. Further, even in one image, aberration varies between the vicinity of the center of the image and the vicinity of four corners of the image, and thus image aligning is difficult.

Examples of a method for solving the problems include a method of arranging a reference point such as markers on a substrate. In this case, it is necessary to determine one position using a combination of multiple points including bright points and reference points. In order to deal with displacement caused by various factors, typically, many reference points such as markers are required. In order to detect these reference points and to determine positions thereof, a load of image processing tends to increase.

In addition, in PTL 2, in order to solve the problem, some spot area is deleted, and this deleted spot area is used as position information to correct displacement. However, samples are not attached to all the attachment spots. Therefore, it is difficult to distinguish between the deletion area of the spot and an attachment spot to which the sample is not attached. Further, the presence of the deletion portion leads to a decrease in sample density.

In nucleic acid analysis, 1,000,000 nucleic acids can be attached in one image, and nearly 500,000 images may be acquired in one analysis. Therefore, erroneous detection of sample positions for arrangement analysis may cause the occurrence of a large number of times of misleading. Therefore, a substrate for nucleic acid analysis and an image aligning technique capable of rapidly aligning images with high accuracy is required.

An object of the present invention is to provide a substrate for nucleic acid analysis capable of arranging samples with high density and aligning the acquired images with high accuracy, a flow cell for nucleic acid analysis, and an image aligning method.

### Solution to Problem

In order to achieve the object, there are provided a substrate for nucleic acid analysis and a flow cell for nucleic acid analysis, the substrate including: a substrate; and a patterned spot area and a randomly distributed spot area that are provided on a surface of the substrate and to which a biopolymer is attached.

In addition, in order to achieve the object, there is provided

an analysis method for a substrate including a patterned spot area and a randomly distributed spot area that are provided on a surface of the substrate and to which a biopolymer is attached, the analysis method including:

identifying bright point positions on the substrate using light-emitting bright points of the patterned spot area and light-emitting bright points of the randomly distributed spot area on the surface of the substrate.

### Advantageous Effects of Invention

According to the present invention, due to the presence of the patterned spot area and the randomly distributed spot area, samples can be arranged with higher density than in a substrate including only the randomly distributed spot area.

In addition, the improvement of the aligning accuracy and speed that is difficult to achieve with only the patterned spot area can be achieved. In the substrate including only the patterned spot area, attachment spots are periodically arranged. Therefore, an adjacent spot array may be erroneously recognized, and large displacement may occur. However, in the substrate where the patterned spot area and the randomly distributed spot area are present, randomly distributed bright points that are detected function as markers or the like. As a result, various positional relationships such as a positional relationship between the patterned spot area and the randomly distributed spot area, a positional relationship between the patterned spot area and randomly distributed bright points, a positional relationship between bright point in the patterned spot area and bright points in the randomly distributed spot area, or a positional relationship between randomly distributed individual bright points can be used without providing special markers for position detection. By using one or a combination of positional relationships depending on usage states, sample position information can be identified with high accuracy. As a result, for example, effects of improving the aligning accuracy and the processing speed can be obtained.

In addition, since a step of providing special markers for position detection is not present, efficient substrate manufacturing can also be expected.

Further, the attachment spot deletion portion described in PTL 2 that functions as a reference point for image aligning is not present. Therefore, attachment spots can be arranged with higher density than that in a case where the spot deletion portion is present.

This way, according to the present invention, the image aligning accuracy can be improved, misreading during sequence analysis of different nucleic acid adjacent to each other can be prevented, and the sequencing accuracy and the throughput of analysis can be improved.

### BRIEF DESCRIPTION OF DRAWINGS

FIG. 1 is a diagram illustrating a schematic configuration example of a nucleic acid analyzer.

FIG. 2 is a diagram illustrating the schematic configuration example of the nucleic acid analyzer.

FIG. 3 is a cross-sectional view illustrating a substrate in a substrate preparation method example.

FIG. 4 is a diagram illustrating a configuration example of a flow cell for nucleic acid analysis.

FIG. 5 is a diagram illustrating an example of a nucleic acid analysis method using the nucleic acid analyzer.

FIG. 6 is a diagram illustrating a concept of a base sequence determination method.

FIG. 7 is a diagram illustrating an arrangement example of a patterned spot area and a randomly distributed spot area.

FIG. 8 is a diagram illustrating an example of a graphical region of the randomly distributed spot area.

FIG. 9 is a diagram illustrating an example of four types of fluorescent images.

FIG. 10 is a diagram illustrating a concept of displacement between cycles.

FIG. 11 is a diagram illustrating an example of an image aligning method.

FIG. 12 is a diagram illustrating an arrangement example of the randomly distributed spot area when one image is divided into 64 blocks.

FIG. 13 is an enlarged view illustrating four blocks of FIG. 12 where one image is divided into 64 blocks.

FIG. 14 is an enlarged view illustrating four blocks of one image when the image is divided into 64 blocks and each of the blocks is further divided into 16 blocks.

FIG. 15 is a diagram illustrating an example of the image aligning method.

FIG. 16 is a diagram illustrating an arrangement example of a patterned spot area, a randomly distributed spot area and attachment spots in the randomly distributed spot area.

FIG. 17 is a diagram illustrating the image aligning method.

### DESCRIPTION OF EMBODIMENTS

Hereinafter, an embodiment of the present invention will be described with reference to the accompanying drawings. For easy understanding of the present invention, a specific embodiment will be described but is not intended to limit the present invention. In addition, for description of the embodiment, nucleic acid analysis refers to sequencing (base sequence analysis) of nucleic acids, that is, DNA fragments. Originally, the analysis target may be a biopolymer such as DNA, RNA, or protein and is applicable to general bio-related materials.

First, a schematic configuration of a nucleic acid analyzer, a method of preparing a substrate for nucleic acid analysis, a flow cell configuration, and a sequencing process of a base sequence of DNA common to the embodiment will be described as an example.

(1) Nucleic Acid Analyzer

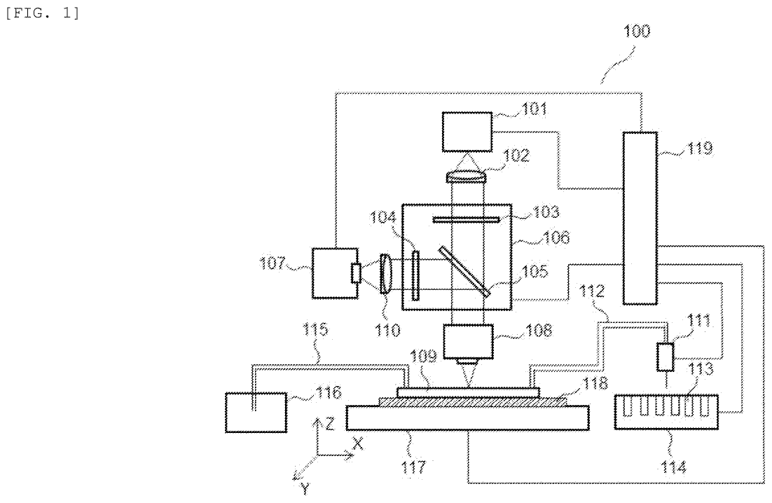

The summary of the nucleic acid analyzer used in the present invention will be described as an example with reference to FIG. 1.

The nucleic acid analyzer 100 includes a flow cell 109, an optical unit, a temperature control unit, a liquid supply unit, and a computer 119.

The optical unit emits exciting light to the flow cell 109 and detects fluorescence emitted from a base sequence incorporated through a nucleic acid extension reaction. The optical unit includes a light source 107, a condenser lens 110, an excitation filter 104, a dichroic mirror 105, a band pass filter 103, an objective lens 108, an imaging lens 102, and a two-dimensional sensor 101. The excitation filter 104, the dichroic mirror 105, and the band pass filter 103 are included in a filter cube 106. The temperature control unit is provided in a stage 117, includes a temperature control substrate 118 that includes, for example, a Peltier element and can execute heating and cooling, and can control the temperature of the flow cell 109. The liquid supply unit includes: a reagent storage unit 114 that accommodates a plurality of reagent containers 113; a nozzle 111 that accesses the reagent container 113; a pipe 112 that introduces each of the reagents in the plurality of reagent containers 113 into a flow cell 109; a waste solution container 116 that disposes of a waste solution such as a reagent after a reaction in the flow cell 109; and a pipe 115 that introduces the waste solution into a waste solution container 116.

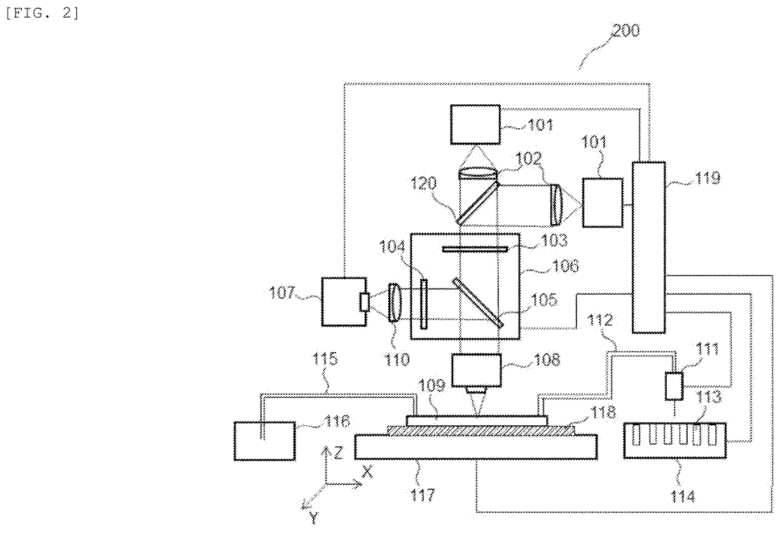

In the nucleic acid analyzer, the flow cell 109 where nucleic acid samples are immobilized in advance is mounted on the stage 117 that is driven in a XY direction. The flow cell has a flow path hole and is fixed to the stage through a vacuum chuck. As a result, the flow cell is connected to a flow path of the liquid supply unit connected to the stage and can supply a solution such as a reaction reagent. A reagent rack 114 is stored in a state where it is kept at a cool temperature and can access the reagent when the nozzle 111 is inserted into the rack. The nozzle is connected to a flow path. Through the operation of a syringe pump, the reagent is finally supplied to a waste solution tank 116 through the flow cell. A plurality of reagents are used, and a reagent to be used is selected by a flow path switching valve. The temperature control substrate 118 is mounted on a XY stage, and a sequence reaction is executed. In the optical unit, for example, a LED light source is used as the light source 107. Exciting light emitted from the light source 107 is condensed by a condenser lens 110 to be incident on the filter cube 106. In the filter cube, the excitation filter 104, the band pass filter 103, and the dichroic mirror 105 are provided, and a specific fluorescence wavelength is selected by the excitation filter 104 and the band pass filter 103. Light transmitted through the excitation filter is reflected from the dichroic mirror 105, and the reflected light is emitted to the flow cell 109 by the objective lens 108. Among fluorescent substances incorporated into samples immobilized on the flow cell 109, a fluorescent substance to be excited in a wavelength range of the emitted exciting light is excited by the exciting light. Fluorescence emitted from the excited fluorescent substance transmits through the dichroic mirror 105, only fluorescence in a specific wavelength range transmits through the band pass filter 103, and the transmitted fluorescence is imaged as fluorescent spots on the two-dimensional sensor 101 by the imaging lens 102. Even one type or plural types of fluorescent substances excited by the exciting light can be detected. For example, when one type of fluorescent substance is excited by the exciting light, the fluorescent substance can be detected by preparing four types of filter cubes 106 corresponding to wavelength ranges to be detected in order to distinguish between four types of fluorescence corresponding to a base sequence and switching between the four filter cubes 106. In addition, FIG. 2 shows a summary example of a nucleic acid analyzer when plural types of fluorescent substances are excited simultaneously, for example, when two types of fluorescent substances are excited simultaneously. A nucleic acid analyzer 200 includes a dichroic mirror 120 that divides fluorescence transmitted through the band pass filter 103 into two types of fluorescence and can execute imaging using a dual view with two two-dimensional sensors, the band pass filter 103 allowing transmission of two types of fluorescence in target wavelength ranges. Four types of fluorescence can be detected by preparing two types of filter cubes 106 corresponding to wavelength ranges to be detected are prepared and switching between the filter cubes 106. In this case, the detection can be executed within a shorter period of time than that in a case where the detection is executed per type, which leads to a reduction in time required to analyze a base sequence of a target sample. In the computer 119, device control and real-time image processing are executed.

(2) Method of Preparing Substrate for Nucleic Acid Analysis, Configuration thereof, and Configuration of Flow Cell

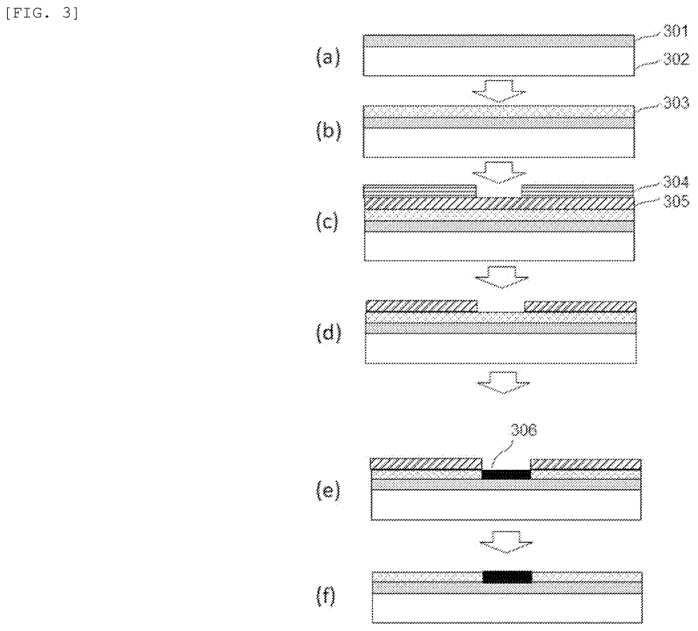

Next, an example of the method of preparing the substrate for nucleic acid analysis used in the present invention will be described with reference to FIG. 3.

First, a heat treatment is executed on a silicon wafer 302 to form an oxide film 301 on a surface of the silicon wafer 302 (FIG. 3-A). The oxide film is coated with a HMDS (Hexamethyldisilizane) layer 303 that is hydrophobic and prevents adsorption of DNA or the like (FIG. 3-B). Next, a protective film is coated, and a photomask 304 where a patterned or randomly distributed spot area is cut out is placed (FIG. 3-C). The protective film 305 is made easily soluble through photolithography, and a development process is executed (FIG. 3-D). Further, the HMDS layer in the spot area is removed by oxygen plasma, and aminosilane 306 or the like is deposited on the removed area as a material for immobilizing a sample (FIG. 3-E). Finally, the protective film is removed by cleaning, and the substrate is prepared (FIG. 3-F).

The material used for the substrate is not particularly limited. For example, when DNA is analyzed with fluorescence or when the temperature is increased or decreased during analysis, silicon, glass, quartz, SUS, titanium or the like in which autofluorescence is low, the thermal expansion coefficient is low, and resistance to an analysis solution is high is particularly desirable.

As a material used for a sample attachment area such as an attachment spot, a material with which the sample attachment area can be formed on the substrate through a covalent bond is preferable. When an inorganic material such as silicon, glass, quartz, sapphire, ceramic, ferrite, or alumina or a metal material such as aluminum, SUS, titanium, or iron including an oxide film on the surface of the substrate is used as the material, a silane coupling material is particularly preferable. In addition, it is preferable that the silane coupling material has a functional group having high reactivity with which a coating film having an amino group through a covalent bond can be formed. For example, ethoxysilane or methoxysilane having, as this functional group, a vinyl group, an epoxy group, a styryl group, a methacryl group, an acrylic group, an amino group, a ureido group, an isocyanate group, an isocyanurate group, or a mercapto group in the molecule is preferable.

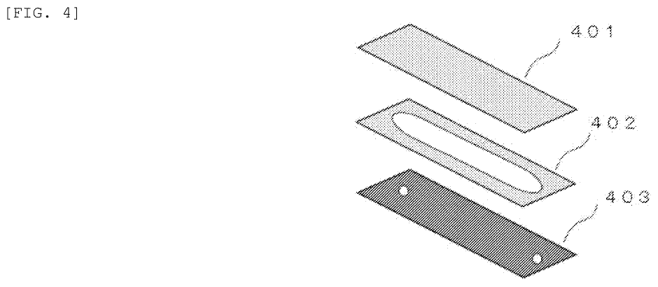

Next, the configuration of the flow cell will be described with reference to FIG. 4.

In the flow cell, a substrate 403 for nucleic acid analysis is provided on a bottom surface, a glass portion 401 is provided on a top surface, and an intermediate material 402 that forms a flow path is interposed between the substrate 403 and the glass portion 401. A hole of the substrate on the bottom surface functions as an injection port and a discharge port of the reagent to be supplied.

(3) Sequencing Process of Base Sequence of DNA

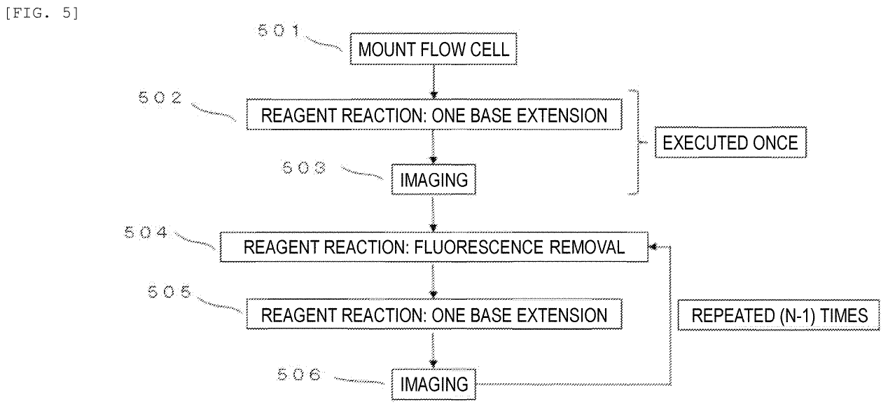

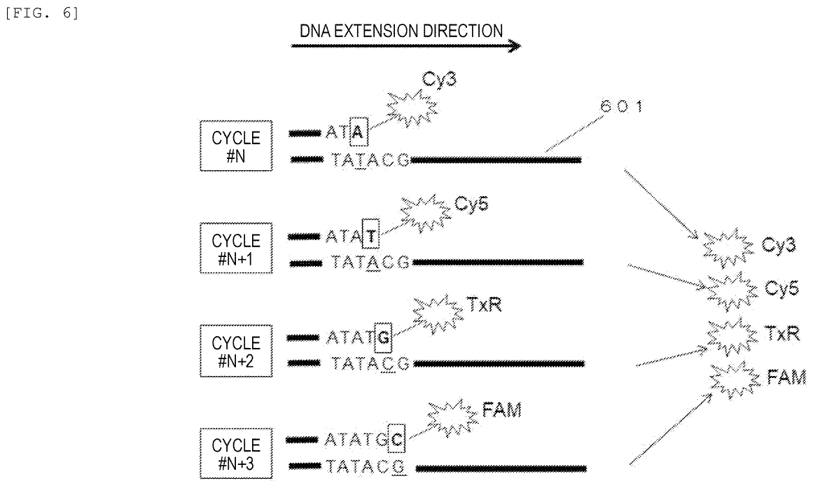

Next, an example of a DNA sequencing method using the nucleic acid analyzer will be described with reference to FIG. 5. First, the flow cell on which DNA as an analysis target is immobilized is mounted on the nucleic acid analyzer 501. Next, a reaction reagent including fluorescence-labeled nucleotides or DNA polymerases where four types of bases are labeled with four different types of fluorescent substances is supplied to the flow cell, the temperature of the flow cell is controlled, and the reagent is caused to react 502. As a result, due to the presence of the base sequence called a primer bound to a sample in advance, nucleotide to which complementary fluorescent substances are attached is incorporated into a sequence of the sample DNA, and an extension reaction is executed. In the nucleic acid analyzer, the type of the incorporated base can be detected by four types of fluorescence. Four bases of A (adenine), T (thymine), G (guanine), and C (cytosine) corresponding to the sequence of the sample DNA as an analysis target can be distinguished from each other. During the fluorescence detection corresponding to the base sequence, four types of fluorescent images are acquired by imaging after cleaning whenever one base is extended 503. Next, the imaged fluorescent substance of one base is removed by a reagent including an enzyme or the like 504. After cleaning, in order to detect the next one base, the previous reaction reagent including fluorescence-labeled nucleotides where fluorescent substances are labeled is supplied to the flow cell, the temperature of the flow cell is controlled, a base reagent to which fluorescent substances are attached is caused to react 505. After cleaning, imaging is executed 506. The fluorescent dye removal, the one base extension, and the imaging 506 are set as one cycle, and this cycle is repeated (N−1) times. As a result, N bases can be sequenced. FIG. 6 shows an example of this sequencing method. In a case where Cy3-dATP, Cy5-dTTP, TxR-dGTP, and FAM-dCTP are used as the fluorescence-labeled nucleotides where fluorescent substances are labeled, when one base is extended by a chemistry treatment in one cycle (#M) in each of attachment spots (for example in a DNA fragment (601) having a base sequence of TATACG), for example, Cy3-dATP as the fluorescent substance is incorporated. This fluorescence-labeled nucleotide is observed as a bright point and is detected as a spot on the fluorescent image of Cy3 during imaging. When the Cy3-dATP is incorporated, the base of the corresponding DNA fragment is determined to be T (thymine). Likewise, in a cycle (#M+1), the fluorescence-labeled nucleotide is observed as a bright point and is detected as a spot on the fluorescent image of the fluorescent substance Cy5. When the Cy5-dTTP is incorporated, the base of the corresponding DNA fragment is determined to be A (adenine). Likewise, in a cycle (#M+2), the fluorescence-labeled nucleotide is observed as a bright point and is detected as a spot on the fluorescent image of the fluorescent substance TxR. When the TxR-dGTP is incorporated, the base of the corresponding DNA fragment is determined to be C (cytosine). Likewise, in a cycle (#M+3), the fluorescence-labeled nucleotide is observed as a bright point and is detected as a spot on the fluorescent image of the fluorescent substance FAM. When the FAM-dCTP is incorporated, the base of the corresponding DNA fragment is determined to be G (guanine). In a cycle treatment from the cycle #M to the cycle #M+3, the base sequence of this spot is determined as TACG. This way, the base sequence of the DNA fragment as a sample is sequenced.

### Embodiment 1

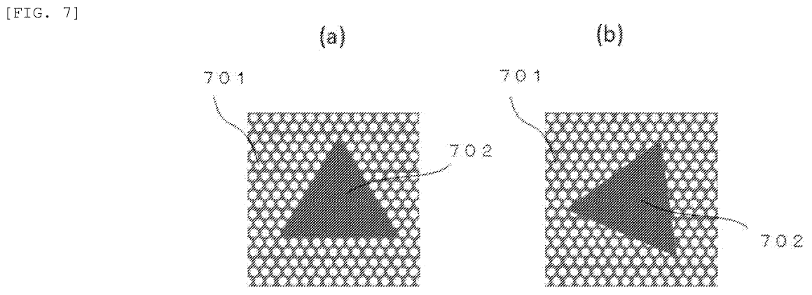

An example of a substrate for nucleic acid analysis including a patterned spot area and a randomly distributed spot area to which nucleic acids are attached on a surface of the substrate will be described with reference to FIG. 7.

FIG. 7 is an enlarged view illustrating a part of the substrate. On the substrate, a patterned spot area 701 as a region where nucleic acid attachment spots are arrayed with certain regularity and a randomly distributed spot area 702 as a region where nucleic acids are attached irregularly are present. In FIG. 6-A, an area where circular portions are arrayed represents the patterned spot area 701, and the circular portion represents an attachment spot to which a sample is attached. A triangular area represents the randomly distributed spot area 702. Each spot area has an area to which a nucleic acid formed of a coating film having an amino group is attached, and the surface of a region to which a nucleic acid is not attached is coated with hydrophobic HMDS. In the patterned spot area, nucleic acids are attached to the arrayed circular portions, a nucleic acid is not attached to the vicinity of the circular portions, and the surface is coated with hydrophobic HMDS. The triangular randomly distributed spot area is formed of a coating film having an amino group to which a nucleic acid is attached.

Here, the patterned form of the spot area where the spots are arranged in a patterned form is an arrangement pattern such as a rhombic lattice pattern, a rectangular lattice pattern, a centered rectangular pattern, a hexagonal lattice pattern, or a square lattice pattern. In particular, it is desirable that attachment spots are arranged in a hexagonal pattern capable of increasing the density of attachment spots. In addition, when the graphic of the randomly distributed spot area is a graphic having sides, it is desirable that each of the sides of the graphic of the randomly distributed spot area is parallel to a spot array of an outer patterned form of the graphic. For example, when the graphic of the randomly distributed spot area is a triangle as illustrated in FIG. 7, it is desirable that each side of the triangle of the randomly distributed spot area does not overlap a patterned attachment spot array positioned in the vicinity of the side as illustrated in FIG. 7-A as compared to a case where a part of the sides of the triangle of the randomly distributed spot area overlaps a patterned attachment spot array positioned in the vicinity of the side as illustrated in FIG. 7-B. Alternatively, it is desirable that each side of the triangle of the randomly distributed spot area is parallel to a patterned attachment spot array positioned in the vicinity of the side. This is because a decrease in the number of spots on a detectable fluorescent image caused by the overlapping between the patterned attachment spots and the randomly distributed spot area can be avoided. In addition, image aligning can also be executed by using a parallel spot array on the outer side of the graphic or spots on the outer circumference of the graphic as an index. For example, a region to be aligned can be selected based on a region positional relationship between the patterned spot area and the randomly distributed spot area, and aligning can be executed by checking a small number of spot positions. As a result, for example, effects of improving the aligning accuracy and the processing speed can be obtained.

In addition, when the graphic of the randomly distributed spot area is a graphic having a circular portion, it is also desirable that the graphic does not overlap the patterned attachment spot array. When the graphic does not overlap the patterned attachment spot array, it is easy to determine the graphic portion of the randomly distributed spot area.

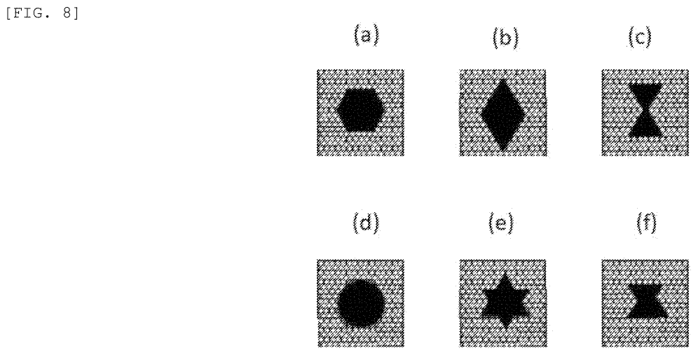

In addition, as illustrated in the examples of FIGS. 8-A, B, C, D, E, and F, as the shape of the randomly distributed spot area, a polygonal shape such as a triangular shape or a quadrangular shape, a circular shape, an elliptical shape, or a graphic including a combination thereof can be considered. In particular, a diagram including a combination of a plurality of triangles has an advantage in that it is easy to distinguish between the patterned area and the randomly distributed area and to use for graphic alignment.

In addition, the randomly distributed spot area can be used as a marker due to a random positional relationship of samples attached to the randomly distributed spot area. Therefore, it is desirable that a plurality of samples are attached without overlapping each other. Therefore, the size of the randomly distributed spot area cannot be stipulated because it varies depending on the sizes of samples. The size of the randomly distributed spot area may be any value as long as a plurality of samples of which positions can be distinguished from each other based on the shape of the region or spot positions in at least each randomly distributed spot area can be attached in the size.

In the patterned spot area, when plural types of nucleic acid samples are attached to one attachment spot, fluorescent dyes are detected from the plural types of nucleic acid samples, and erroneous detection occurs. Therefore, when the size of the attachment spots is excessively large, erroneous detection may occur. On the other hand, when the size of the attachment spots is excessively small, the probability of contact with nucleic acid samples decreases, the number of attachment spots to which a nucleic acid sample is not attached increases, and the throughput of analysis decreases. Therefore, regarding the diameter of the patterned attachment spots or the arrangement of the attachment spots, it is desirable that the size or the position is determined such that only one nucleic acid sample is attached to one attachment spot, and the size of the attachment spot is ½ or more and less than 2 times with respect to the size of a sample. In this case, an excellent result can be obtained. For example, when the nucleic acid sample has a size of 50 nm, it is desirable that the size of the attachment spot is 25 nm or more and less than 100 nm.

### Embodiment 2

An example of image acquisition and an aligning method using the substrate for nucleic acid analysis including the patterned spot area and the randomly distributed spot area will be described.

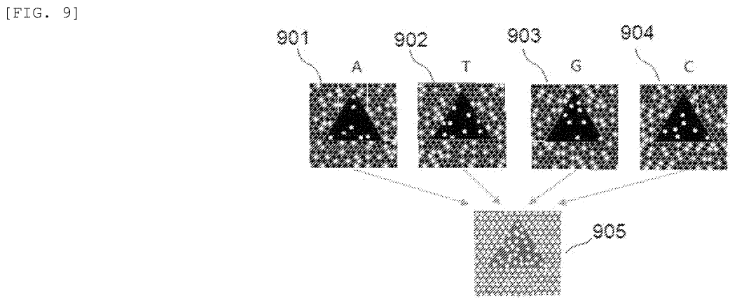

Nucleic acid samples as analysis targets are immobilized in the patterned spot area and the randomly distributed spot area arranged in the substrate on the flow cell. Through an extension reaction, nucleotides to which fluorescent substances are attached are incorporated, four types of fluorescent images corresponding to four types of DNA bases are acquired by imaging. In each cycle of one base extension, four types of fluorescent images are observed as bright points per field of view. FIG. 9 illustrates an example of the four types of fluorescent images. White circles represent the bright points. The bright points can be detected as spots on the fluorescent images. Bright point positions of an image 905 obtained by combining images (901, 902, 903, and 904) corresponding to the four types A, T, G, and C represent positions where nucleic acid samples per image are immobilized.

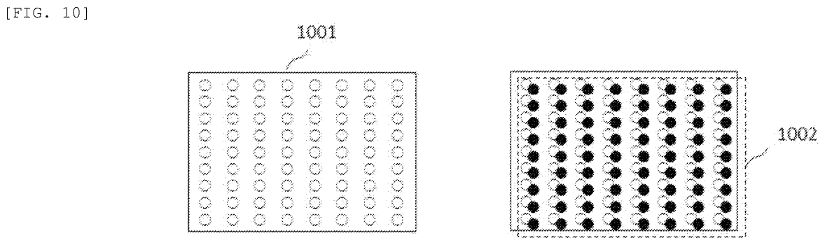

In addition, the number of detection field of views where fluorescent images of the substrate are detected varies depending on the size of the substrate or the resolution of the analyzer and may be several hundreds. For example, when the number of detection field of views is 800, the stage is moved by 800 field of views for imaging in each cycle. As illustrated in FIG. 10, there may be a displacement between a cycle N(1001) and a cycle N+1(1002) due to the movement of the stage. This displacement occurs due to various factors such as the control accuracy of stage driving or the distortion of the substrate caused by heat.

In order to analyze nucleic acid samples, it is necessary to repeat steps of incorporating nucleotides to which fluorescent substances are attached through an extension reaction and acquiring bright point images to acquire bright point position information using the substrate where the nucleic acid samples are immobilized. In order to analyze nucleic acids using a plurality of images, it is necessary to accurately align the plurality of images.

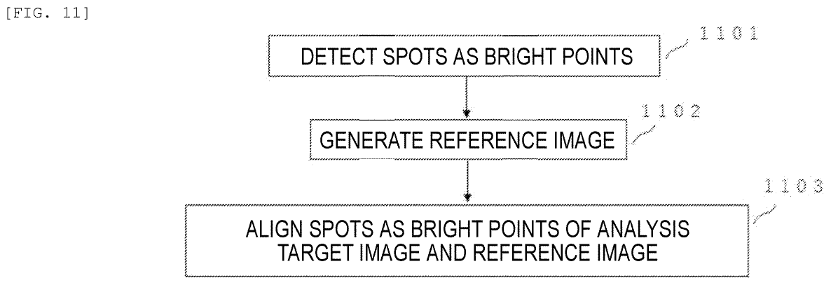

An example of the image aligning method will be described using FIG. 11. First, all the spots on the fluorescent images as bright points are detected 1101. Next, a reference image as a reference for aligning is generated 1102. Here, the reference image refers to an image of positions of spots as a reference used for aligning position coordinates of spots on fluorescent images as bright points. The positions of the spots as bright points of the analysis target image and the reference image are aligned with respect to the positions of the spots as bright points of the reference image 1103.

The reference image (K1) is generated based on the acquired actual image. For example, in the case of nucleic acid analysis, four bright point images based on base types of four types of nucleobases ATCG are acquired per field of view in each cycle. Initially, four images in the first cycle are combined to generate the reference image (K1). In the four images acquired per field of view in the first cycle, when there is no stage movement, there is no displacement that may be caused by the stage movement. Therefore, it is easier to superimpose the images as compared to a case where there is a stage movement.

Unless a plurality of samples are attached to spots on one fluorescent image, the spots on the respective fluorescent images as bright points do not overlap each other on the four images. Therefore, the images are superimposed such that the spots on the respective fluorescent images do not overlap each other. For example, when the images of FIG. 9 are the fluorescent images (901, 902, 903, and 904) corresponding to the four types of A, T, G, and C acquired in the first cycle, the combined image 905 is the reference image (K1).

In addition, when a special primer with which all the bright points can be detected by imaging is used, one fluorescent image where all the bright points are detected can also be used as the reference image.

In addition, the reference image (K1) may be generated by combining fluorescent images acquired in a plurality of cycles. In this case, portions to which samples are attached are bright points corresponding to the base sequences of the respective samples, and the bright points are detected as spots on the fluorescent image. Therefore, in order to align bright point positions, while repeating rotation, scaling, and translation of the images, the spots on the respective fluorescent images may be aligned using a method capable of minimizing the square of the distance between spots on the respective fluorescent images. When the same spots are identified, by combining a plurality of images acquired in a plurality of cycles, the accuracy can be improved and erroneous detection can be prevented. Even when a plurality of samples are attached to one spot, the samples can be distinguished from each other. However, when the number of images to be used is excessively large, a long period of time is required to calculate aligning, and the throughput decreases.

In addition, the reference image generated based on the four images in the first cycle may be corrected based on four images acquired in the next cycle or may be corrected based on images acquired in a plurality of cycles. For example, by aligning images in the second cycle to the tenth cycle and the initial reference image (K1), the reference image (K1) is corrected to generate a reference image (K2). Images in the eleventh cycle may be aligned using this reference image (K2).

In addition, the reference image may be corrected as the error of image aligning increases or at regular time intervals. By correcting the reference image, a deviation in stage driving caused by imaging in a plurality of cycles or a plurality of field of views or a temporal change such as substrate distortion caused by heat or the like can be handled.

Further, during the preparation of the reference image or the aligning of the reference image and the analysis target image, the bright points of the patterned spot area are easily aligned because the attachment spots are arrayed regularly. On the other hand, when the bright points are erroneously recognized as an adjacent array, displacement may occur. On the other hand, the coordinates of the bright point positions in the randomly distributed spot area are random. Therefore, the bright points can be used as position markers based on a positional relationship between the plurality of bright points and are useful for aligning bright points. Therefore, by correcting the bright points in the randomly distributed spot area after aligning the bright points in the patterned spot area, displacement can be avoided. In addition, when the bright points are aligned with the aligning method using the bright points in the randomly distributed spot area, the region of the randomly distributed spot area according to the present invention is smaller than that of a substrate including only randomly distributed spots, and thus aligning can be executed within a short period of time. This way, with the substrate including both the patterned spot area and the randomly distributed spot area, by executing detection for aligning using a combination of superior characteristics of the patterned spot area and superior characteristics of the randomly distributed spot area, aligning can be easily executed, and the throughput of analysis can be improved. In addition, by providing both regions of the patterned spot area and the randomly distributed spot area, positions of the regions can be estimated based on the arrangement of the respective regions. In addition, the bright point positions can be also identified simply by aligning the bright point positions of the randomly distributed spot area.

In addition, when the alignment among images is performed, images can be aligned in units of blocks by dividing one image into a plurality of blocks in order to improve the aligning accuracy or speed. By dividing the area to be aligned into small blocks and executing aligning in units of blocks, the number of bright points for executing aligning is reduced, and the aligning speed increases. In this case, a decrease in the number of bright points refers to a decrease in the number of bright points as markers for aligning, and it may be difficult to identify the block units. The positions of the block units can be identified based on bright point position information of surrounding blocks. In this case, it is desirable that at least one patterned spot area and at least one randomly distributed spot area are present in each of the blocks. However, when each of the block positions can be distinguished based on a positional relationship of surrounding blocks, a block including no randomly distributed spot area may be present.

The number of blocks divided from one image is not limited. For example, when the randomly distributed spot areas have the same positional relationship periodically on the substrate, it is desirable that the size of unit blocks is larger than the size of image displacement occurring during observation.

When the size of unit blocks is larger than the size of image displacement, by searching blocks to be matched in the vicinity of a target block to be aligned, the position of the target block can be identified. On the other hand, when the size of unit blocks is smaller than the size of image displacement, it is necessary to increase the number of blocks to be searched according to the size of image displacement.

In addition, in an image acquired by imaging, aberration varies between the center of the screen and four corners of the screen. Therefore, when image aligning is executed, the amount of displacement also varies. Therefore, as the number of randomly distributed spot areas increases, the aligning accuracy increases. By randomly arranging randomly distributed spot areas on the substrate, not only the bright point positions of randomly distributed spots but also an arrangement pattern of the randomly distributed spot areas can be imparted with uniqueness, which may contribute to aligning in a well-known arrangement.

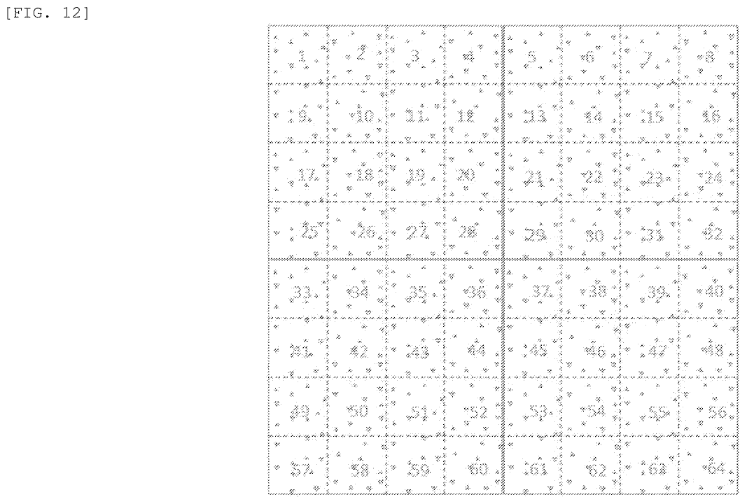

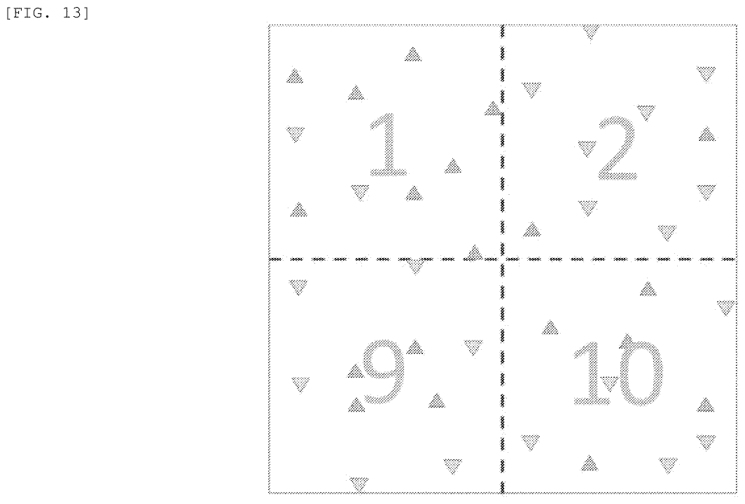

When the size of unit blocks is larger than the size of image displacement occurring during observation, an example of the arrangement pattern of the randomly distributed spot areas and the divided blocks will be described below. For example, FIG. 12 illustrates a case where one image is divided into 64 blocks assuming that the size of one image is about 1 mm² and the size of image displacement is within about 0.1 mm. FIG. 12 illustrates one image which is divided into 64 blocks. For convenience of description, the respective unit blocks are assigned with numbers 1 to 64, but the numbers may be removed. The respective blocks are arranged such that the arrangements of the randomly distributed spot areas in at least blocks adjacent to each other are different. In addition, in order to easily design or manufacture the substrate, for example, the arrangements of the randomly distributed spot areas in all the 64 blocks do not have to be different from each other, and the same arrangement may be used per four unit blocks. FIG. 13 is an enlarged view illustrating blocks 1, 2, 9, and 10 of FIG. 12 as an example of the four unit blocks. The four unit blocks have different arrangements of the randomly distributed spot areas. By arranging 16 block units for each of the four unit blocks, 64 blocks in total may be arranged. With this arrangement method, effects of easily manufacturing the substrate and reducing the costs can be obtained.

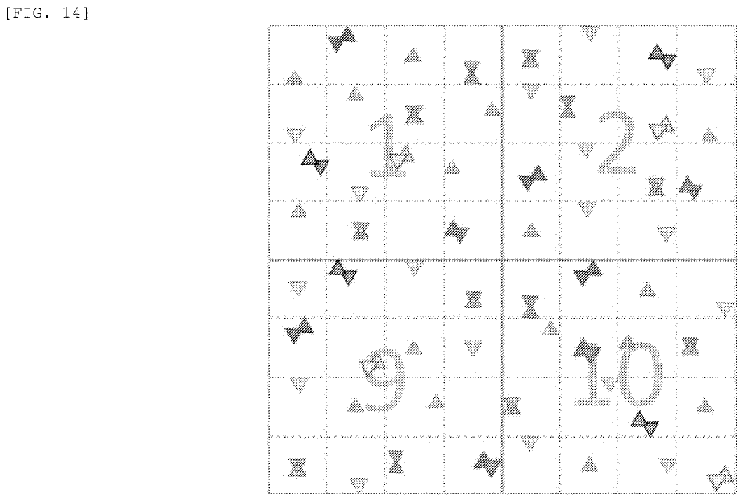

In addition, an example of further dividing the above-described unit blocks into smaller blocks will be described using FIG. 14. In FIG. 14, by increasing the types and the number of the randomly distributed spot areas, the uniqueness of the block units is improved. FIG. 14 illustrates an example in which one image is divided into 64 blocks and each of the 64 blocks is further divided into 16 blocks. 4/64 blocks of one image are illustrated. In order to identify the positions of the blocks based on the arrangements of the surrounding randomly distributed spot areas, it is preferable that at least one randomly distributed spot area is arranged in each of the divided blocks. In addition, when the block positions in the arrangement of the randomly distributed spot area can be identified based on the arrangements of the surrounding randomly distributed spot areas, there may be a block including no randomly distributed spot area among the divided blocks.

FIGS. 12, 13, and 14 illustrate only the randomly distributed spot area without illustrating the patterned spot area.

### Embodiment 3

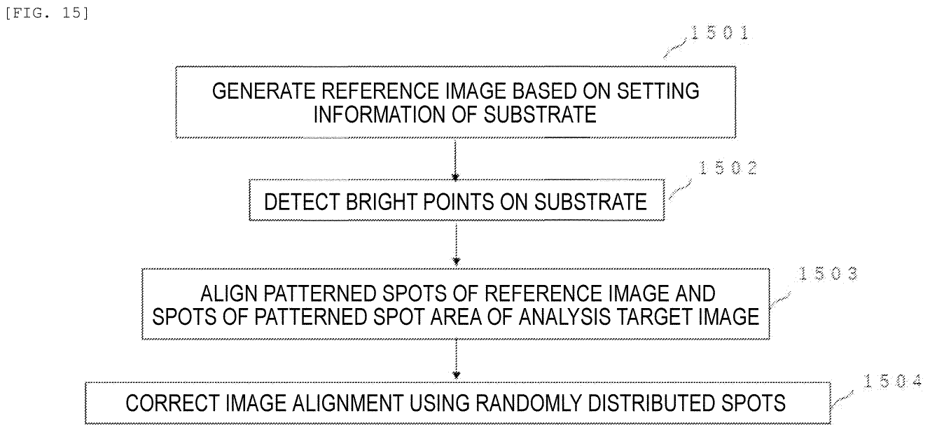

FIG. 15 illustrates an example of the image aligning method.

The reference image is generated based on substrate design information 1501. For example, the reference image may be generated through simulation or the like. Here, the reference image refers to an image of positions of spots as a reference used for aligning position coordinates of spots on fluorescent images as bright points. When only spot position information is combined, the image does not need to be generated. The reference image may be generated in advance depending on the substrate to be used. The reference image generated in advance may be read from a storage medium depending on the substrate to be used. The initial reference image generated based on the substrate design information shows the positions of the attachment spots in the patterned spot area. Depending on use conditions, the initial reference image may include a region of the patterned spot area or region information of the randomly distributed spot area. Next, the bright points on the substrate are detected 1502. The bright points on the substrate are detected as spots on the fluorescent image. Next, the positions of the patterned spots in the analysis target image are aligned with respect to the positions of the spots in the patterned spot area of the reference image 1503. The aligning of the patterned spot area has an advantage in that the reference image of which position information is already known is present. Therefore, high-throughput aligning can be implemented. However, in the aligning of the patterned spot areas, the spots are periodically aligned. Therefore, an adjacent spot array may be erroneously recognized. Therefore, image aligning is corrected using the spots on the fluorescent image as the bright points of the randomly distributed spot area, that is, using the randomly distributed spots 1504. Regarding the randomly distributed spots, the distance between adjacent spots is irregular. Therefore, it is easier to determine the positions of all the randomly distributed spots than in the patterned spot area. The bright point position information of the randomly distributed spot area is not included in the reference image of the position information of the patterned spot area generated based on the substrate design information. Therefore, the reference image is corrected using the bright point information acquired in each cycle.

### Embodiment 4

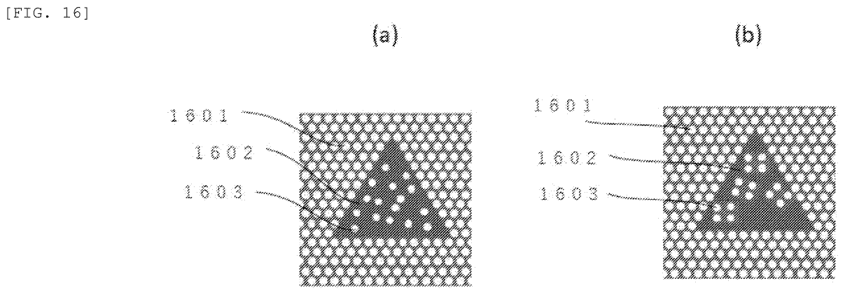

Another example different from Embodiment 1 regarding the substrate for nucleic acid analysis including the patterned spot area and the randomly distributed spot area to which nucleic acids are attached on the surface of the substrate will be described with reference to FIG. 16.

FIG. 16 is an enlarged view illustrating a part of the substrate. On the substrate, a patterned spot area 1601 as a region where nucleic acid attachment spots are arrayed with certain regularity and a randomly distributed spot area 1602 where attachment spots to which nucleic acids are attached are arranged irregularly are present. The randomly distributed spot area 1602 includes attachment spots 1603 that are irregularly arranged in the randomly distributed spot area. Each of the attachment spots is formed of a coating film having an amino group, and a nucleic acid can be attached thereto. The surface of a region to which a nucleic acid is not attached is coated with hydrophobic HMDS. In the patterned spot area, nucleic acids are attached to arrayed circular portions. In the randomly distributed spot area, likewise, nucleic acids are attached to circular portions. A nucleic acid is not attached to the vicinity of the circular portion, and the surface of the circular portion is coated with hydrophobic HMDS. The attachment spots in the randomly distributed spot area are arranged during the preparation of the photomask 304 described in the above-described example of the method of preparing the substrate for nucleic acid analysis. The arrangement of the attachment spots in the randomly distributed spot area is an arrangement in which the spots are not in contact with each other and is different from the that of a surrounding patterned spot area. The arrangement where the attachment spots are irregularly arranged represents that the arrangement is different from the regular arrangement of the surrounding patterned spot area, and represents that the arrangement is different from the arrangement of the surrounding patterned attachment spots when one attachment spot or a plurality of attachment spots are compared to the surrounding patterned attachment spots. In addition, although depending on the number or density of the attachment spots to be arranged, the respective spot positions may be identified based on the spot positions on the fluorescent image in the randomly distributed spot area and the spot positions in the randomly distributed spot area or based on the spot positions in the randomly distributed spot area and the spot positions in the patterned spot area. FIG. 16-(A) illustrates an example in which the attachment spots alone are randomly arranged in the randomly distributed spot area. FIG. 16-(B) illustrates an example in which aggregates of the attachment spots having a positional relationship different from that of the attachment spots in the patterned spot area are randomly arranged. FIG. 16-(B) illustrates the example where a plurality of aggregates of four attachment spots are arranged. The aggregates of the attachment spots may adopt any number or any arrangement but, desirably, can be distinguished from at least the patterned spot area in order to be used as position markers.

### Embodiment 5

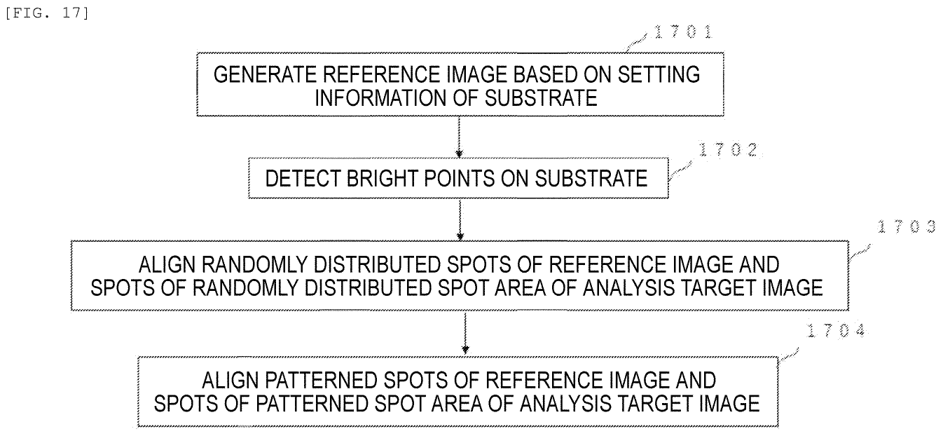

FIG. 17 illustrates an aligning method relating to images when the substrate according to Example 4 is used.

A reference image of position information of each spot area is generated based on the substrate design information 1701. For example, the reference image may be generated through simulation or the like. Here, the reference image refers to an image of positions of spots as a reference used for aligning position coordinates of spots on fluorescent images as bright points. When aligning with only spot position information, the image does not need to be generated. The reference image may be generated in advance depending on the substrate to be used. The reference image generated in advance may be read from a storage medium depending on the substrate to be used. The initial reference image generated based on the substrate design information is generated based on the positions of the attachment spots in the patterned spot area and the positions of the attachment spots in the randomly distributed spot area. In order to align the acquired images, the initial reference image may include a region of the patterned spot area or region information of the randomly distributed spot area. Next, the bright points on the substrate are detected 1702. The bright points on the substrate are detected as spots on the fluorescent image. Next, the positions of the spots in the randomly distributed spot area of the reference image and the positions of the spots in the randomly distributed spot area of the analysis target image are aligned 1703. The aligning of the randomly distributed spot area has an advantage in that the reference image of which position information is already known is present, and the area to be aligned is small. Therefore, high-throughput aligning can be implemented. In addition, regarding the randomly distributed spots, the distance between adjacent spots is irregular. Therefore, it is easier to determine the positions of all the randomly distributed spots than in the patterned spot area. Next, the spots of the patterned spot area and the patterned spots of the analysis target image are aligned 1704. Since aligning of the randomly distributed spot area is executed, there is an advantageous effect in that the spots of the patterned spot area can be easily aligned.

The present invention is not limited to the embodiment described above and includes various modification examples. For example, the embodiments have been described in detail in order to understand the present invention, and the present invention is not necessarily to include all the configurations described above. In addition, addition, deletion, and replacement of another configuration can be made for a part of the configuration of each of the embodiments.

### REFERENCE SIGNS LIST

100: nucleic acid analyzer

101: two-dimensional sensor

102: imaging lens

103: band pass filter

104: excitation filter

105: dichroic mirror

106: filter cube

107: light source

108: objective lens

109: flow cell

110: condenser lens

111: nozzle

112: pipe

113: reagent container

114: reagent rack

115: pipe

116: waste solution container

117: stage

118: temperature control substrate

119: computer

120: dichroic mirror

200: nucleic acid analyzer

301: oxide film

302: silicon wafer

303: HMDS

304: photomask

305: protective film

306: aminosilane

401: glass plate

402: intermediate material

403: substrate

501: mount flow cell

502: reagent reaction: one base extension

503: imaging

504: reagent reaction: fluorescence removal

505: reagent reaction: one base extension

506: imaging

601: base sequence of DNA fragment

701: patterned spot area

702: randomly distributed spot area

901: image emitted from fluorescent nucleotide corresponding to A (adenine)

902: image emitted from fluorescent nucleotide corresponding to T (thymine)

903: image emitted from fluorescent nucleotide corresponding to G (guanine)

904: image emitted from fluorescent nucleotide corresponding to C (cytosine)

905: image obtained by superimposing 901 to 904

1001: stage position in cycle N

1002: displacement caused by stage movement in cycle N+1

1101: detect bright points of spots

1102: generate reference image

1103: align positions of bright points of analysis target image and reference image

1501: generate reference image based on substrate design information

1502: detect bright points on substrate

1503: align positions of patterned spots of reference image and patterned spots of analysis target image

1504: correct image aligning using randomly distributed spots

1601: patterned spot area

1602: randomly distributed spot area

1603: attachment spot

1701: generate reference image based on substrate design information

1702: detect bright points on substrate

1703: align positions of randomly distributed spots of reference image and positions of randomly distributed spots of analysis target image

1704: align positions of patterned spots of reference image and patterned spots of analysis target image

1. A substrate for nucleic acid analysis comprising:
a substrate; and
a patterned spot area and a randomly distributed spot area that are provided on a surface of the substrate and to which a biopolymer is attached.

2. The substrate for nucleic acid analysis according to claim 1,
wherein the randomly distributed spot area is configured with a graphical region, and
a plurality of samples are randomly arranged in the randomly distributed spot area.

3. The substrate for nucleic acid analysis according to claim 1,
wherein in the patterned spot area, spots to which a sample is attached are regularly arranged.

4. The substrate for nucleic acid analysis according to claim 2,
wherein the graphical region of the randomly distributed spot area is formed of a coating film to which a sample is attachable.

5. The substrate for nucleic acid analysis according to claim 2,
wherein spots to which a sample is attached are irregularly arranged in the graphical region of the randomly distributed spot area.

6. The substrate for nucleic acid analysis according to claim 3,
wherein the patterned spot area has a patterned arrangement where spots to which a sample is attached are arranged in a hexagonal lattice pattern.

7. The substrate for nucleic acid analysis according to claim 2,
wherein the graphical region of the randomly distributed spot area is arranged not to overlap the patterned spots.

8. A flow cell for nucleic acid analysis comprising:
a substrate including a patterned spot area and a randomly distributed spot area that are provided on a surface of the substrate and to which a biopolymer is attached;
a glass member that covers a top surface of the substrate; and
a sheet as an intermediate material that forms a flow path.

9. An analysis method for a substrate including a patterned spot area and a randomly distributed spot area that are provided on a surface of the substrate and to which a biopolymer is attached, the analysis method comprising:
identifying bright point positions on the substrate using light-emitting bright points of the patterned spot area and light-emitting bright points of the randomly distributed spot area on the surface of the substrate.

10. The analysis method according to claim 9, comprising the following steps of:
generating a reference image to execute image aligning using the reference image and images of bright points of the patterned spot area; and
correcting image aligning using bright points of the randomly distributed spot area.

11. The analysis method according to claim 9, comprising:
a step of generating the reference image using four bright point images based on nucleobase types; and
a step of correcting the reference image using a plurality of images.

12. The analysis method according to claim 9, comprising a step of generating the reference image using position information of each spot to which a sample is to be attached during preparation of the substrate.

13. The analysis method according to claim 9, comprising a step of aligning the reference image and an analysis target image using a numerical value with which a square of a distance between bright points on each of the analysis target image and spots corresponding to the reference image is the minimum.

14. The analysis method according to claim 9, comprising a step of dividing one image into a plurality of blocks such that at least one patterned spot area and at least one randomly distributed spot area are present.
